(19) United States
(12) Patent Application Publication
(10) Pub. No.: US 2001/0036282 A1
(43) Pub. Date: Nov. 1, 2001

(54) Active noise attenuation inlet microphone system

(76) Inventors: Roy Haworth, Shrewsbury (GB); John F. Astorino, Livonia, MI (US)

Correspondence Address: LAURA M. SLENZAK, SIEMENS CORPORATION, 186 WOOD AVENUE SOUTH, ISELIN, NJ, 08830, US

(21) Appl. No.: 09/847,245

(22) Filed: May 2, 2001

Related U.S. Application Data
(60) Provisional application No. 60/203,780, filed on May 12, 2000.
(60) Provisional application No. 60/203,776, filed on May 12, 2000.
(60) Provisional application No. 60/203,777, filed on May 12, 2000.
(60) Provisional application No. 60/203,779, filed on May 12, 2000.

(51) Int. Cl. A61F 11/06; G10K 11/16; H03B 29/00; H04R 9/08

(52) U.S. Cl. 381/71.5; 381/71.7; 381/361; 381/366; 181/224

(57) ABSTRACT

The air induction system comprises an air induction body having a mouth. A speaker and microphone are both operatively connected to the air induction body. A support connects the microphone to the air induction body. The support may be rigid and extend over only a portion of the face of the speaker. The support may also be spaced a predetermined distance from the speaker face. This distance may relate to the location of the sound field emitted by the speaker. Both the speaker and microphone are in communication with a control unit. The microphone receives sound and communicates this sound to the control unit. The control unit then generates a noise attenuation sound through the speaker.

This application claims priority to Provisional Patent Application Ser. Nos. 60/203,780 filed May 12, 2000, 60/203,776 filed May 12, 2000, 60,203,777 filed May 12, 2000, and 60/203,779 filed May 12, 2000.

### BACKGROUND OF THE INVENTION

This invention relates to an active method and system for controlling automotive induction noise.

Manufacturers have employed active and passive methods to reduce engine noise within the passenger compartment. Such noise frequently emanates from the engine, travels through the air induction system and emanates out of the mouth of the air intake into the passenger compartment. Efforts have been made to reduce the amount of engine noise traveling through the air induction system. These efforts include the use of both passive devices such as expansion chambers and Helmholtz resonators and active devices involving anti-noise generators.

Active systems use a speaker to create a canceling sound that attenuates engine noise. The sound created is out of phase with the engine noise and combines with this noise to result in its reduction. Generally, this sound is generated in proximity to the mouth of the air induction system. In one such system, a control unit, such as a digital signal processor, obtains data from the vehicle engine, creates a predictive model of engine noise, and thereby generates the appropriate cancellation signal based on the results of this model. This signal is then transmitted to the speaker, which transforms this signal into a canceling sound. Because the control unit may not perfectly model engine noise, an error microphone is placed in proximity to the mouth of the air induction system to determine if engine noise need be further attenuated.

Typically, the error microphone is placed on the fender of the vehicle while the speaker of the system is generally attached to the air induction system. During vehicle operation, road conditions may cause the error microphone to vibrate significantly. Because the speaker is located on the air induction system, the speaker and error microphone may vibrate at different rates. As a consequence, the noise attenuation system may function less than optimally due to the relative movement between the speaker and error microphone. The noise attenuation system may even generate a howling sound or other undesirable noise as a consequence of this problem.

One proposed solution is the attachment of the error microphone to a piece of open cell foam, which is then connected to the housing of the speaker. The foam is mounted on the speaker. While foam permits the error microphone to vibrate with the speaker, it has several drawbacks. Foam is not durable, serving as a less than optimal support for an error microphone over the operational life of the vehicle. In particular, foam is prone to damage caused by weathering, road salt, road wear, and other environmental conditions, which are encountered during the operation of a vehicle.

Moreover, the foam support covers the entire speaker face. As a consequence, the foam support creates acoustic interference for the noise attenuation system under certain frequencies and even restricts airflow around the air intake. Also, because the foam support is mounted to the speaker face, it may interfere with the speaker's operation.

A need therefore exists to provide for a support that permits the error microphone to vibrate with the speaker of the noise attenuation system without the drawbacks of a foam microphone support.

### SUMMARY OF THE INVENTION

The invention comprises an air induction body having a mouth. A speaker is connected to the air induction body. Rather than a foam support, a rigid support is used to connect the microphone to the air induction body, locking the microphone in place and providing a durable connection to the air induction body. The rigid support may be made of plastic. Both the microphone and speaker are in communication with a control unit.

The microphone may be operatively connected to the mouth of the air induction body. A screen may span at least a portion of the mouth of the air induction system. This screen, which may be nylon, serves to protect the air induction system from large objects that may find their way in the path of the moving vehicle.

The rigid support is designed to be as acoustically transparent as is reasonably possible. One design of the invention comprises a rigid support with at least one leg operatively connecting the microphone to the air induction body. This particular design may comprise a plurality of legs radially extending from the microphone, thereby connecting the microphone to the air induction body. The design may also be vented by one or a plurality of openings that may skirt the mouth of the air induction system.

Another aspect of the invention comprises an air induction body, having a mouth. A speaker is connected to the air induction body and has a face. A support extending over only a portion of the face of the speaker connects the microphone to the air induction body. A control unit communicates with the speaker and the microphone.

The microphone may be connected to the mouth by the support. The support is also designed to be acoustically transparent and may be made of plastic. A screen may span a portion of the mouth. The support may comprise at least one leg operatively connecting said microphone to said air induction body. A plurality of legs may radially extend from the microphone, connecting it to the air induction body. A ring may extend circumferentially to connect the plurality of rings.

The invention may also comprise an air induction body, having a mouth and a speaker, having a face. The microphone is connected to the air induction body by a support spaced a predetermined distance from the face wherein the predetermined distance relates to the location of the sound field emitted by the speaker. A control unit is in communication with the speaker and the microphone. The support may be designed to be acoustically transparent.

By employing this invention, the microphone and speaker are mounted together in a reliable manner. The invention covers only a portion of the speaker face, greatly reducing any acoustic interference or airflow restriction caused by the microphone support. The support also does not interfere with speaker operation. Hence, the invention permits microphone and speaker to vibrate together during vehicle operation, improving noise attenuation and durably holding the microphone in place.

### BRIEF DESCRIPTION OF THE DRAWINGS

The various features and advantages of this invention will become apparent to those skilled in the art from the following detailed description of the currently preferred embodiment. The drawings that accompany the detailed description can be briefly described as follows:

FIG. 1 shows a schematic view of an embodiment of the invention, including speaker, microphone, and support.

FIG. 2 shows a perspective view of the embodiment of FIG. 1, including speaker, microphone, support, and air induction body and mouth.

FIG. 3 shows a side view of the embodiment of FIGS. 1-2, illustrating speaker, microphone, support, and mouth.

FIG. 4 shows a front view of the support of the embodiment of FIGS. 1-3 without microphone.

FIG. 5 shows a side view of the embodiment of FIGS. 1-4.

FIG. 6 shows an internal view of the embodiment of FIGS. 1-5, including speaker and air inlet.

FIG. 7 shows the embodiment of FIGS. 1-6 with a screen.

FIG. 8 shows a perspective view of another embodiment of the invention.

FIG. 9 shows a side view of an embodiment of the invention, including microphone.

FIG. 10 shows a front view of the embodiment of FIG. 9, including microphone.

### DETAILED DESCRIPTION OF THE PREFERRED EMBODIMENT

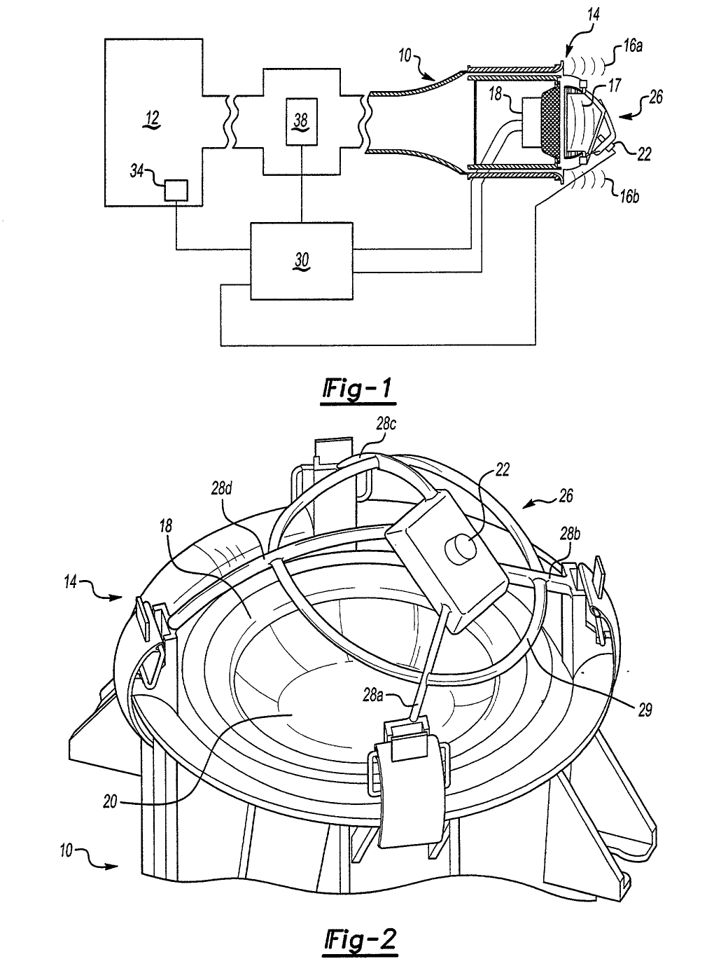

FIG. 1 illustrates an embodiment of the invention. Shown are air induction body 10 with mouth 14. Speaker 18 with face 20 is operatively connected to air induction body 10 by a support as known. Microphone 22, an error microphone, is operatively connected by rigid support 26 to air induction body 10 instead of to a fender of a vehicle. The material used to construct rigid support 26 is not pliable like foam, allowing rigid support 26 to firmly lock microphone 22 into position.

Plastic is one such material that may be used for rigid support 26. Rigid, as used in this application, should be interpreted relative to the non-rigid open cell foam of the prior art. Both microphone 22 and speaker 18 are in communication with control unit 30. Also in communication with control unit 30 are engine speed sensor 34 and throttle position sensor 38. These sensors 34 and 38 provide information on engine speed and position of throttle blade as taught by the prior art.

As known, engine noise 16A and 16B emanate from vehicle engine 12 through air induction 10 out of mouth 14. Speaker 18 generates sound field 17 with sound out of phase with engine noise 16A and 16B. While it is known to place microphone 22 about sound field 17, a novel feature of the invention concerns the spacing away of support 26 from speaker 18 while still operatively connecting microphone 22 to air induction body 10 through support 26. Hence, this embodiment of the invention involves microphone 22 operatively connected to air induction body 10 by support 26, which is spaced a predetermined distance from face 20 of speaker 18 wherein the predetermined distance relates to the location of sound field 17 emitted by speaker 18. It is preferable to locate microphone 22 off the center of sound field 17 to ensure complete reception of sound. Support 26 is also preferably designed to place microphone 22 off center of sound field 17 as shown.

FIG. 2 illustrates a perspective view of a portion of the embodiment pictured in FIG. 1. FIG. 2 shows microphone 22 set in rigid support 26. Rigid support 26 is operatively connected to mouth 14, which itself is part of air induction body 10. As known in the art, speaker 18 is disposed in mouth 14. Rigid support 26 extends over only a portion of face 20 and may comprise at least one leg such as 28A, 28B, 28C, and 28D operatively connecting microphone 22 to air induction body 10. An encircling ring 29 extends circumferentially to connect legs 28A-28D. The design of rigid support 26 is thus designed to be relatively acoustically transparent to avoid interference with low frequency sound waves. Acoustic transparency is accomplished by ensuring that rigid support 26 does not itself create a pressure barrier during operation of the system.

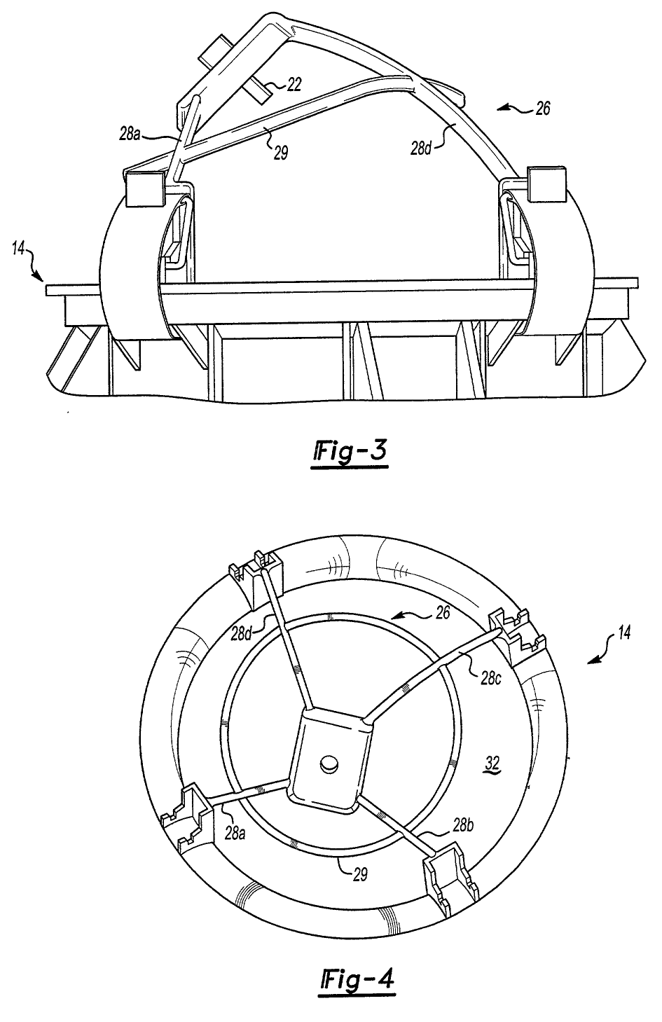

FIG. 3 shows a side view of the embodiment of FIGS. 1 and 2. Microphone 22 is more clearly shown locked in rigid support 26. Rigid support 26 is operatively connected to mouth 14.

FIG. 4 shows a front view of the embodiment of FIGS. 1-3. Rigid support 26 and mouth 14 are shown. FIG. 4 also illustrates a plurality of legs 28A, 28B, 28C, and 28D radially extending from the microphone and operatively connecting the microphone to air induction body 10, here at mouth 14. Rigid support 26 is also vented by at least one opening 32. This design is relatively acoustically transparent at low frequencies and creates only negligible air restriction around mouth 14. Not pictured is microphone 26.

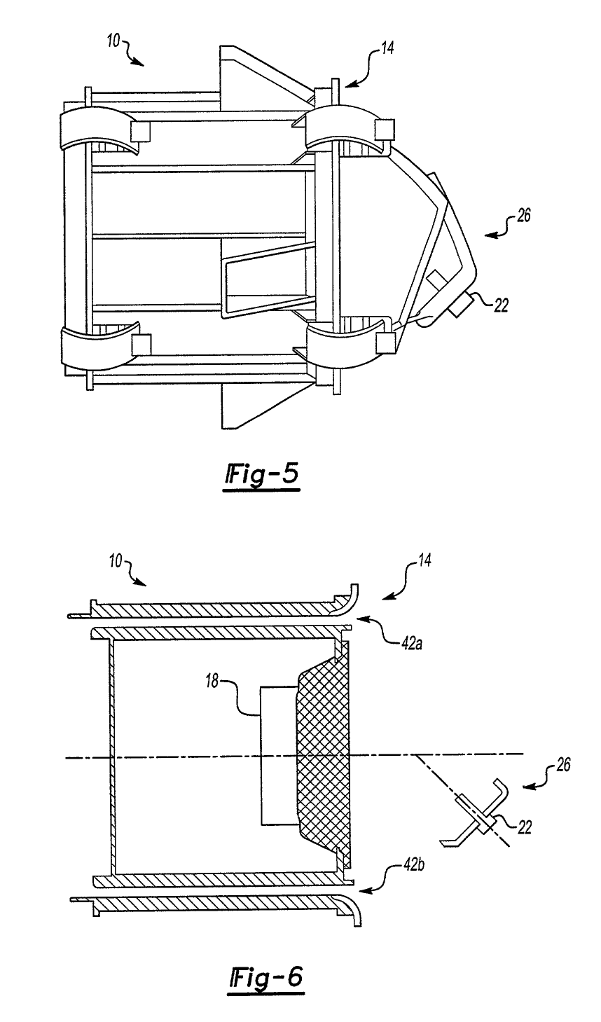

FIG. 5 shows a side view of the embodiment of FIGS. 1-4. Rigid support 26, mouth 14, and microphone 22. Speaker 18 is hidden in this perspective.

FIG. 6 shows a side view of the embodiment of FIG. 5 with speaker 18 shown. Also illustrated are air induction body 10, mouth 14, microphone 22, and a portion of rigid support 26. As known, air flows into air induction body 10 through inlets 42A and 42B.

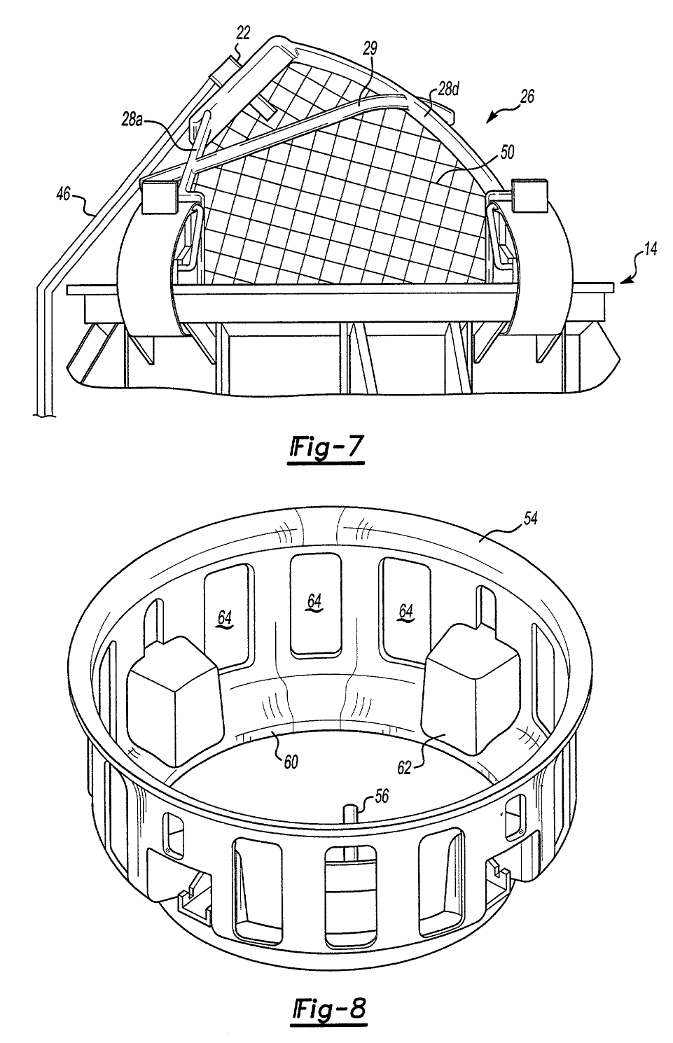

FIG. 7 shows another side view of the embodiment of FIG. 5 with another feature. Mouth 14, rigid support 26, and microphone 22 are illustrated.

Also shown is conductor 46, which is operatively connected to rigid support 26 through microphone 22. Conductor 46 may be a flex cable. Moreover, screen 50, provides protection of the air induction system from debris that may otherwise enter system. Screen 50 may be a nylon mesh. Note that the legs 28A-28D and ring 29 preferably provide said positioning and draping for screen. However, other embodiments may use the screen.

FIG. 8 shows a perspective view of another embodiment of the invention.

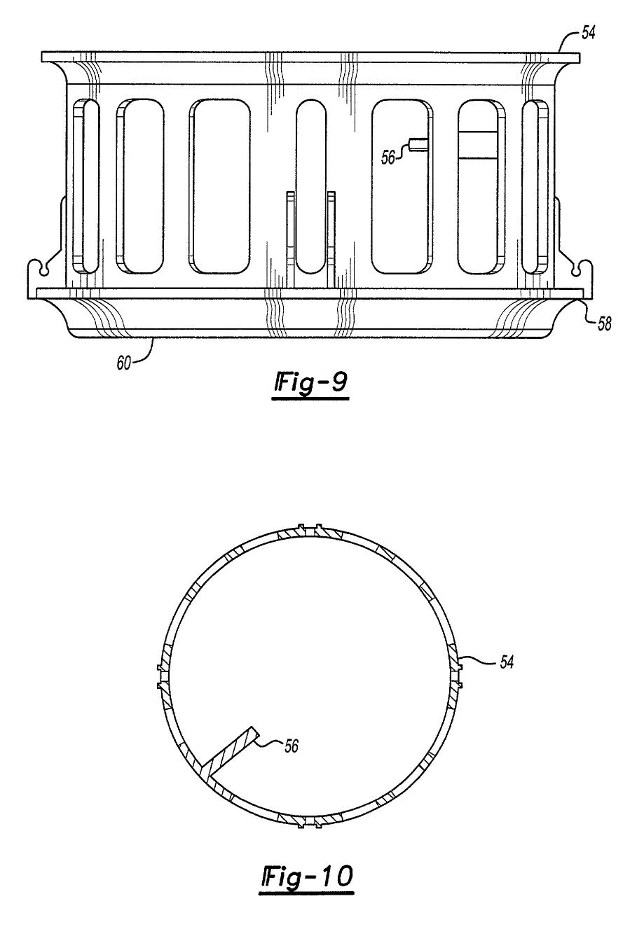

Shown is another rigid support 54. Microphone 56 is also shown. Here, support 54 is vented by a plurality of openings 64 skirting mouth 60. These openings 64 serve to prevent the creation of a pressure barrier around mouth 60 of air induction body 10. In this way, rigid support 54 becomes acoustically transparent. Mounts, such as mount 62, are also shown. Mount 62 is used to quickly connect rigid support 54 to air induction body 10. A side view of this embodiment is shown in FIG. 9. Here, rigid support 54 is operatively connected to mouth 58.

FIG. 10 shows a front view of the embodiment, illustrating the location of microphone 56. Notably, microphone 56 is molded as part of rigid support 54 and located off center of rigid support 54. In this way, microphone 56 is in optimal position to receive sound field 17 (as seen in FIG. 1).

The aforementioned description is exemplary rather then limiting. Many modifications and variations of the present invention are possible in light of the above teachings. The preferred embodiments of this invention have been disclosed. However, one of ordinary skill in the art would recognize that certain modifications would come within the scope of this invention. Hence, within the scope of the appended claims, the invention may be practiced otherwise than as specifically described. For this reason the following claims should be studied to determine the true scope and content of this invention.

What is claimed is:

1. An air induction system comprising:
an air induction body having a mouth;
a speaker operatively connected to said air induction body;
a microphone operatively connected to said air induction body by a rigid support; and
a control unit in communication with said speaker and said microphone.

2. The air induction system of claim 1, wherein said microphone is operatively connected to said mouth.

3. The air induction system of claim 1, wherein a flex cable communicates with said speaker.

4. The air induction system of claim 1, wherein said rigid support is plastic.

5. The air induction system of claim 1, including a screen spanning at least a portion of said mouth.

6. The air induction system of claim 1, wherein said rigid support comprises at least one leg operatively connecting said microphone to said air induction body.

7. The air induction system of claim 6, wherein said rigid support comprises a plurality of legs radially extending from said microphone and operatively connecting said microphone to said air induction body.

8. The air induction system of claim 6, wherein a circumferentially extending ring connects said plurality of legs.

9. The air induction system of claim 8, wherein a screen is draped on said legs and said ring.

10. An air induction system comprising:
an air induction body, having a mouth;
a speaker operatively connected to said air induction body, having a face;
a microphone operatively connected to said air induction body by a support extending over only a portion of said face; and
a control unit in communication with said speaker and said microphone.

11. The air induction system of claim 10, wherein said microphone is operatively connected to said mouth.

12. The air induction system of claim 10, wherein a flex cable communicates with said speaker.

13. The air induction system of claim 10, wherein said support is plastic.

14. The air induction system of claim 10, including a screen spanning at least a portion of said mouth.

15. The air induction system of claim 10, wherein said support comprises at least one leg operatively connecting said microphone to said air induction body.

16. The air induction system of claim 15, wherein said support comprises a plurality of legs radially extending from said microphone and operatively connecting said microphone to said air induction body.

17. The air induction system of claim 10, wherein a circumferentially extending ring connects said plurality of legs.

18. The air induction system of claim 17, wherein a screen is draped on said legs and said ring.

19. An air induction system comprising:
an air induction body, having a mouth;
a speaker operatively connected to said air induction body, having a face;
a microphone operatively connected to said air induction body by a support spaced a predetermined distance from said face wherein said predetermined distance relates to the location of the sound field emitted by said speaker; and
a control unit in communication with said speaker and said microphone.

20. The air induction system of claim 19, wherein said support is designed to be acoustically transparent.
